(19) United States
(12) Patent Application Publication
(10) Pub. No.: US 2026/0341054 A1
(43) Pub. Date: Oct. 29, 2026

(54) GROSS DISSECTION SAMPLE TRACKING SYSTEM

(71) Applicant: MP Acquisition, LLC, Madison Hts., MI (US)

(72) Inventor: Michael Scott McClain, Waterford, MI (US)

(73) Assignee: MP Acquisition, LLC, Madison Hts., MI (US)

(21) Appl. No.: 19/193,564

(22) Filed: Apr. 29, 2025

(51) Int. Cl. G06V 20/69; G06T 7/00; G16H 30/40

(52) U.S. Cl. CPC G06V 20/698; G06T 7/0012; G06V 20/695; G16H 30/40; G06T 2207/30024

(57) ABSTRACT

The present invention relates to a safety and security gross dissection sample tracking system that incorporates a software application in conjunction with a digital camera to track tissue samples during a gross dissection process. The software via the digital camera will identify and track tissue as it is sectioned from the original sample and placed into tissue containers by recognizing unique tissue characteristics, such as cell morphology. The gross dissection sample tracking system may verify and confirm that the tissue sample is properly matched with the correct sample container throughout the dissection process. This gross dissection sample tracking system may provide continuous monitoring of the chain of custody of the tissue sample from the moment of dissection thus ensuring that the tissue remains in its original container. This gross dissection sample tracking system eliminates human error and provides a high level security and sample tracking during dissection procedures.

### BACKGROUND OF THE INVENTION

### 1. Field of the Invention

The present invention relates generally to pathology workflow, and more specifically relates to a gross dissection sample tracking system that includes a software application for use with a gross dissection bench or grossing station.

### 2. Description of Related Art

Pathology workflows, especially in gross dissections have become increasing complex with the advent of new imaging and diagnostic technologies. In pathology and histology laboratories, for example, tissue samples are carefully dissected from larger gross specimens for further analysis. A critical challenge during this process is to ensure that the tissue samples are accurately tracked from the dissection stage through to the final processing stage. Currently, human intervention plays a major role in the tracking of these samples. This may introduce a risk for error and potential mislabeling, thus comprising the integrity of the sample identification.

Many existing methods of tracking tissue samples dissected from larger gross specimens for further analysis rely on manual processes to track these tissue samples during the dissection procedure, thus creating significant opportunities for misidentification or misplacement of these samples. These errors may lead to disruptions in the pathology workflow and errors in patient diagnosis or research outcomes. Therefore, there is a need in the art for a gross dissection bench or grossing station that is more reliable and has an automated tracking system to ensure the integrity of tissue samples during the dissection and subsequent processing stages of that dissected sample. Furthermore, there is also a need in the art for a gross dissection sample tracking system that may be able to eliminate the risk of workflow disruptions and errors by introducing an automated system that tracks and verifies tissue samples both from a dissected gross specimen and the original gross specimen itself in real time, beginning from the very first step of dissection until the final step of diagnosis or research outcomes.

### SUMMARY OF THE INVENTION

One object of the present invention may be to provide an improved grossing station.

Another object of the present invention may be to provide a grossing station that has a gross dissection sample tracking system incorporated therein.

Still another object of the present invention may be to provide a grossing station that has a high resolution digital camera arranged on a surface thereof.

Still another object of the present invention may be to provide a grossing station that has a gross dissection sample tracking software application that works in conjunction with a high resolution digital camera to automate the tracking of the tissue samples during the gross dissection process.

Yet another object of the present invention may be to provide a grossing station that identifies unique tissue characteristics such as shape, cell morphology, etc., during the dissection procedure.

Yet another object of the present invention may be to provide a grossing station that has a gross dissection sample tracking system that is capable of linking specific tissue characteristics to a corresponding tissue container having a unique identification label in real time, thus ensuring that the tissue sample remains in the correct container throughout the dissection process, hence maintaining a precise chain of custody.

Still another object of the present invention may be to provide a grossing station that has a gross dissection sample tracking system that may incorporate machine learning or artificial intelligence (AI) to assist in identifying the dissection pattern for a given tissue sample based on its cell morphology or other factors.

Yet another object of the present invention may be to provide a grossing station that has a gross dissection sample tracking system that via a continuous monitoring and matching of tissue samples to their containers may reduce the risk of human error thus improving sample tracking while enhancing the security and safety of the tissue sample management in pathology laboratories.

According to the present invention, the foregoing and other objects and advantages are obtained by a novel apparatus and methodology that provides a grossing station with a gross dissection sample tracking system. The gross dissection tracking system provides an automated solution to track tissue samples during the gross dissection process. The gross dissection sample tracking system may include a digital camera arranged on a predetermined surface of the grossing station. The digital camera provides a continuous feed. The digital camera feed is then processed by a gross dissection sample tracking software application that may identify and monitor tissue characteristics, such as cell morphology during the dissection procedure. The gross dissection sample tracking system may then link these tissue characteristics to a corresponding tissue containers unique identification label in real time, such as but not limited to, a barcode, 2D matrix or QR code, etc. The gross dissection sample tracking system ensures that the tissue sample remains in the correct container throughout the dissection and processing phases, thus maintaining a precise chain of custody. Furthermore, the gross dissection sample tracking software system may incorporate machine learning or AI to assist in identifying an optimal dissection pattern for a given tissue sample based on its cell morphology. This may allow the sample to be sectioned according to best practices at the current time, wherein the software may continuously verify that the dissected tissue matches the recommended pattern and it is properly tracked in the correct sample container throughout the entire process.

One advantage of the present invention may be that it provides for an improved grossing station.

Another advantage of the present invention may be that it provides for an improved grossing station that uses a gross dissection sample tracking system therewith.

Still another advantage of the present invention may be that it provides for a grossing station that includes a high resolution digital camera arranged on a predetermined surface of the grossing station.

Yet another advantage of the present invention may be that it provides a grossing station that uses a gross dissection sample tracking software application in conjunction with a high resolution digital camera in order to identify and monitor tissue characteristics, such as cell morphology during the dissection procedure.

Yet another advantage of the present invention may be that it provides a gross dissection sample tracking system that is capable of linking tissue characteristics to the corresponding tissues container unique identification label in real time, thus ensuring the tissue sample remains in the correct container throughout the dissection and processing phases, hence maintaining a precise chain of custody.

Yet another advantage of the present invention may be that it provides a grossing station that may incorporate machine learning or artificial intelligence into a gross dissection sample tracking system in order to identify a dissection pattern for a given tissue sample based on cell morphology. This may ensure that the sample is sectioned according to the best practices in the medical field at the current time.

Another advantage of the present invention may be that it provides a grossing station that with the use of a gross dissection sample tracking system may provide continuous monitoring and matching of tissue samples to their appropriate containers, thus reducing the risk of human error while improving sample tracking and enhancing the security of tissue samples managed throughout the pathology laboratory.

Other objects, features and advantages of the present invention may become apparent from the subsequent description, and the appended claims, taken in conjunction with the accompanying drawings.

### BRIEF DESCRIPTION OF THE DRAWINGS

FIG. 1 shows a plan view of a grossing station according to the present invention.

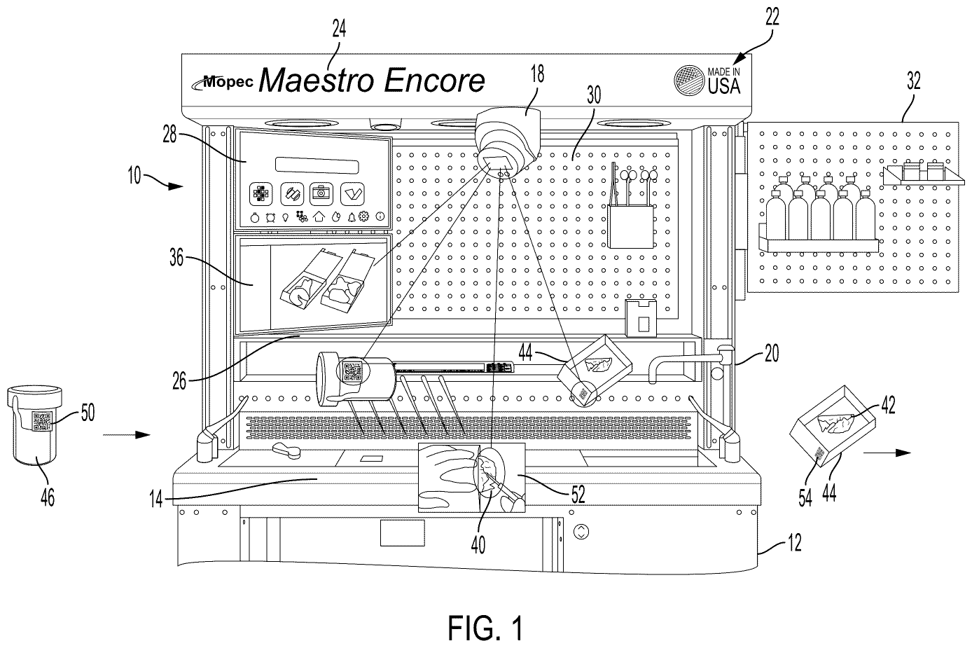

FIG. 2 shows a flowchart for a grossing station having a gross dissection sample tracking system according to the present invention.

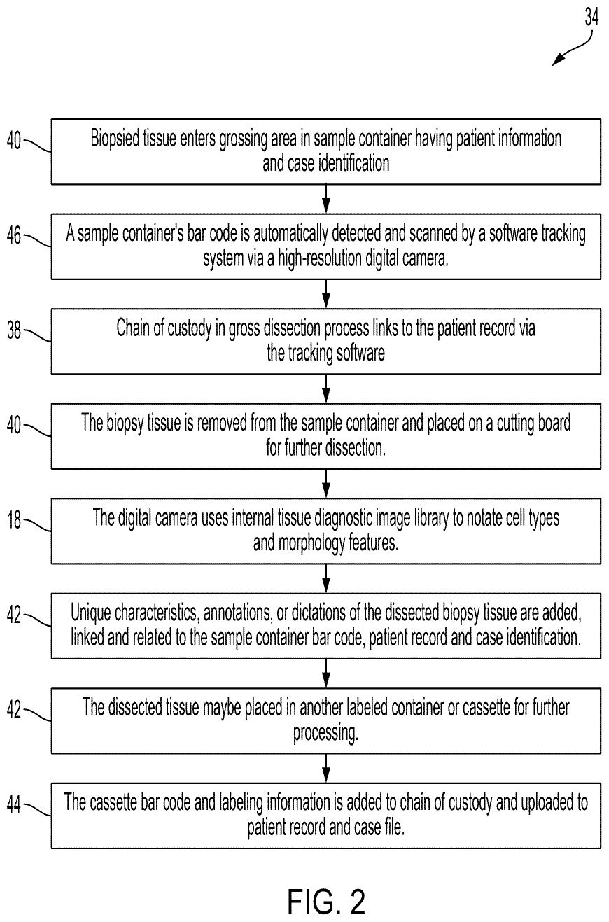

### DESCRIPTION OF THE EMBODIMENT(S)

Referring to the drawings, a grossing station 10 that is used in autopsy and necropsy procedures is shown according to the present invention. It should be noted that the grossing station 10 may be used on any known autopsy, necropsy, trimming, pathology, or any other procedure performed on tissues or the like, all of which are for use in hospitals, morgues, funeral homes, university laboratories, or other laboratories, or any other structures that may be used for examining human or animal bodies or human or animal body parts.

The grossing station 10 of the present invention may include a base 12, which generally is arranged on the floor or another surface in the laboratory or room in which the grossing station 10 may be used. It should be noted that it is also contemplated to attach the grossing station 10 to a wall so it is elevated off of the floor and capable of movement with respect to the floor. The grossing station 10 may also include a table or work surface area 14 arranged over a top portion of the base 12. The table 14 may have a generally rectangular shape and a flat work surface arranged on the top end thereof. The grossing station 10 may have a variety of technologies arranged thereon including but not limited to a sink 16, imaging technologies 18, and other types of grates, panels, or grids, which may be arranged in troughs or on surfaces thereof. A faucet 20 may also be arranged on the table 14 of the grossing station 10 or grossing dissection bench.

The grossing station 10 also may include a hood 22 that may be secured to a top surface of the table 14 of the grossing station 10. The hood 22 may have two side walls and/or partial side walls, a rear wall and a top member 24. The top member 24 when viewed from above, generally has a rectangular shape. It should be noted that any necessary lighting and associated electronics to control the lighting may be arranged within or on the surfaces of the hood 22 or on any surface of the grossing station 10. The rear wall of the hood 22 may also have a plurality of or one shelf 26 arranged thereon for holding supplies, tools, computer equipment 28 or the like necessary for the pathology procedure being performed on the grossing station 10. It is also contemplated that the rear wall of the hood 22 may have a metal peg board 30 to allow for hanging of various components or objects needed or used during pathology procedures. It is also contemplated that a first and second panel 32 may be arranged on each end of the first and second side wall of the hood 22 or the grossing station 10, wherein these panels 32 generally are of a peg board configuration, made out of metal, which may allow for the placement of shelfs in any desired location and/or lights or any other type of material or component needed during the dissection of the material being worked on. It should be noted that many other tools and components may be arranged on or near the grossing station 10 of the present invention. The above components are some of those that may be used on the grossing station 10. Furthermore, the connections and components necessary for the grossing station 10 may be specific to the laboratory needs of the user of the grossing station 10 and the technology or platform arranged thereon. It should further be noted that generally most of the parts of the grossing station 10 are made of a metal material, however any other known material such as but not limited to any known ceramic, composite, plastic, or any other metal or any known manmade or natural material may also be used to make any of the components discussed herein for the grossing station 10 according to the present invention.

The gross dissection sample tracking system 34 of the present invention may also include a hardware platform arranged and integrated onto any surface of the grossing station 10. In one contemplated embodiment, the hardware platform may comprise a mini computer or dedicated computer 36 that is embedded or integrated within the grossing station or gross dissection bench that acts as the host for the software application of the gross dissection sample tracking system 34 of the present invention. It should be noted that the hardware platform may also be in the form of a phone or tablet that is wirelessly connected or wired into the grossing station 10 on any surface thereof.

The gross dissection sample tracking system 34 of the present invention maybe for use on the grossing station 10 as described above or any other known grossing station or table. The gross dissection sample tracking system 34 may include a high resolution digital camera 18 arranged on a predetermined surface of the grossing station 10. In one contemplated embodiment, the high resolution digital camera 18 may be arranged on an underside surface of the hood 22 of the grossing station 10. This may allow for a full view of the work area or table by the high resolution high speed digital camera 18. This digital camera 18 may be capable of either taking still images continuously or recording video in a continuous manner during any gross dissection procedure. It should be noted that the digital camera may have a manual on/off switch or an automated on/off switch. If it is automated when a person or container with a gross dissection sample enters the work area of the grossing station 10, the camera 18 automatically begins recording either video or still images and then may stop recording the still images or videos when the biopsy tissue and/or dissected samples and/or the worker working on the samples leaves the work area. In another contemplated embodiment, the person doing the dissection procedure may manually turn on the high digital camera 18 and then turn off the high resolution digital camera 18 and by default the software application upon leaving the grossing station 10. Therefore, the gross dissection sample tracking system 34 also uses a software application 38 in conjunction with the digital camera 18 to identify and track a dissected tissue sample 42 as it is sectioned from the original biopsy tissue sample 40 and placed into tissue containers. By recognizing unique tissue characteristics, such as but not limited to cell morphology, cell shape, cell size, the software 38 may verify and confirm that the dissected tissue sample 42 is properly matched with the correct sample container 44 throughout the entire dissection process. It should be noted that it also correctly identifies the original biopsy tissue sample 40 with the original sample container 46. This gross dissection sample tracking system software application 38 may provide continuous monitoring, via the high resolution digital camera 18, of the chain of custody of the original biopsy tissue sample 40 and any dissected tissue samples 42 from the initial moment of dissection, thus ensuring the tissue, either the original sample 40 or dissected samples 42 remain in their respective original container 46 or associated dissected sample container 44. This process eliminates reliance and human error and provides a high level of security for the dissected and original sample tissue tracking during the dissection procedures. It should be noted that an original biopsy tissue sample 40 may enter the grossing area in one specimen container 46 and may then be dissected into one or multiple dissected tissue samples 42. The multiple samples 42 then leave the grossing area in multiple dissected tissue sample containers 44. Furthermore, the original biopsy tissue sample 40 may be very large, such as but not limited to many feet of intestine, such that it enters the grossing area in a large dish container or the like that includes accompanying paperwork having barcode tracking information or a case ID number or identification label. This barcode or case ID number will also be read by the system 34 and become associated with the patient and any dissected samples 42 taken from the original biopsy tissue sample 40. It is also contemplated that the gross dissection sample tracking system 34 of the present invention may employ machine learning or artificial intelligence (AI) to identify known cell morphologies and recommend a dissection grid pattern for the original biopsied tissue sample 40, thus ensuring optimal tissue sectioning for the dissection procedures. This gross dissection sample tracking system 34 thus may operate from the initial dissection step and provide continuous confirmation of the tissues location via a matching of the tissue and the container it is in by the software 38 therefore, the overall integrity of the dissection process may be greatly improved.

Therefore, the gross dissection sample tracking system 34 of the present invention uses a gross dissection sample tracking software application 38 in conjunction with the digital camera 18, which generally may be positioned above the gross dissection bench 10. However, it should be noted that the digital camera system may also be placed within the gross dissection bench 10 to provide a feed from below but generally it is preferred to provide a continuous camera feed from above the gross dissection bench 10. This digital camera 18 may also capture a continuous feed of images or video of the dissection process, wherein those images or videos may be processed by the gross dissection sample tracking software application 38 to detect and identify unique features of the original biopsied tissue sample 40 and of the dissected tissue samples 42. These features may include, but are not limited to, the shape, texture or cell morphology of the original biopsy tissue sample 40 and of the dissected tissue samples 42. These features may then be compared against a ready known database having specific tissue characteristics arranged therein. Comparing the dissected tissue samples 42 taken from the original biopsy tissue sample 40 may allow for matching to the tissue characteristics arranged in the known database to help with further dissection needs or views needed of the dissected tissue sample 42, thus creating a more efficient and accurate pathology procedure in determining the final results of the biopsy. Furthermore, when the original biopsy tissue sample 40 enters the grossing area AI may be able to identify known cell morphologies and recommend a dissection sample grid pattern for that original biopsy tissue sample 40 presented into the grossing area. Then at that moment, the first steps of tissue tracking and the chain of custody would begin. Next, confirmation may be made by the software 38 of the gross dissection tracking sample system 34 such that the biopsied original tissue sample 40 was dissected in the recommended pattern. The original biopsied tissue sample 40 may be confirmed to have been dissected to the original recommended pattern or adaptation to what was actually sectioned and finally matching that to the dissected tissue sample containers 44 labeling as scanned from the digital live camera feed of the dissection area.

Therefore, the gross dissection sample tracking system 34 of the present invention provides many improvements and sample tracking right from the beginning of the dissection process that otherwise relies on human interaction in the prior art and becomes a high risk point for dissection workflow errors. The gross dissection sample tracking system 34 of the present invention immediately identifies tissue sectioning shape along with tissue morphology characteristics and then takes these known data points and maps those to the labeling used on the tissue container 44, 46 associated therewith, it may provide the best chain of custody tracking available at step one of a very long tissue process and procedure in the pathology laboratory. The gross dissection sample tracking system 34 of the present invention may ensure that throughout the entire dissection procedure, either the original biopsy tissue sample 40 or the dissected tissue sample or samples 42 from that original biopsy tissue sample 40 chain of custody is preserved with all relevant data logged and continuously updated via the gross dissection sample tracking system 34. This provides an unprecedented level of security and accuracy in the tissue dissection process.

In operation, the gross dissection sample tracking system 34 of the present invention starts when a dissection process begins by having a biopsied original tissue sample 40 enter the grossing area or grossing station 10 of the present invention in an original sample container 46. This original sample container 46 may have a barcode or other 2D data matrix 50 arranged thereon, which may include but not be limited to patient information, case identification, etc., for that original biopsy tissue sample 40. Thus, the patient information and the case identification may be automatically associated with the original biopsy sample tissue 40 and uploaded into the patient record and case file automatically via the gross dissection sample tracking system 34, which comprises a high resolution digital camera 18 and a software application 38 associated therewith. Once that original biopsy tissue sample 40 enters the grossing area, the original sample container barcode 50 may be automatically detected via the high resolution digital camera 18 and scanned by the associated software tracking system via that high resolution digital camera 18. Then at that very point, the initial chain of custody within the gross dissection process may begin and may link to the patient record via the gross dissection sample tracking software application 34. The transfer of data to the patient health record or EHR system from the grossing station 10 occurs on either a wired or wireless connection. From that point on within the patient record and case file that original biopsied sample tissue 40 is identified to that original sample container 46. The original biopsied tissue sample 40 may be removed from the original sample container 46 and placed on a cutting board 52 or other surface for further dissection. At that point, the digital camera 18 via the associated gross dissection sample tracking software application 38 may utilize feature recognition from its internally loaded tissue diagnostic image library to note any cell types and any other cell morphology features that are within the field of view of the high definition camera 18. These identified unique characteristics and any other annotations or dictations that may be added by the pathologist or pathologist assistants are then linked in real time and related to the original sample container barcode 50 and in conjunction the patient record and case number. Next, pathologists may dissect a tissue piece from the original biopsied sample tissue 40 and either typically place it in another dissected labeled container 44 or back into the original sample container 46. However, most commonly it is placed in another dissected labeled container 44 that has a unique barcode 54 associated therewith. In some cases, the dissected label container 44 may be a tissue cassette as shown in the drawings, however any other known dissected label container 44 may also be used. This dissected label container 44 may then be used after being associated with the patient record for further processing and histology. Therefore, at the time the dissected tissue 42 is cut from the original biopsied tissue sample 40 and placed in either the original sample container 46 or another dissected label container 42 the barcode 54 of the dissected labeled container 44 and its associated labeling information is added to the chain of custody of documentation of the original biopsied tissue sample 40 and uploaded into the patient record and case file via the gross dissection sample tracking system software 38. Thus, any dissected sample or samples 42 taken from the original biopsied tissue sample 40 is either placed in the same original sample container 46 or into its own dissected label container that have been associated with the original sample container thus keeping the chain of custody automated and updated in real time. The gross dissection sample tracking system 34 of the present invention provides an innovative solution to ensure the security, integrity and accurate tracking of original biopsied tissue samples 40 and/or a dissected tissue sample or samples 42 from the very beginning of the gross dissection process. Hence, the system 34 may be used with a single dissected tissue sample 42 and the associated single dissected labeled container 44 or with a plurality of dissected tissue samples 42 and the associated plurality of dissected labeled containers 44. This is done by leveraging real time image analysis, machine learning and continuous monitoring, wherein the associated software of the gross dissection sample tracking system 34 eliminates the risk of human error and provides the best possible chain of custody throughout the pathology and histology laboratory workflow.

The gross dissection sample tracking system 34 provides a high digital camera 18, along with a safety and security software application 38 designed to track tissue samples 40,42 during the gross dissection process. This gross dissection sample tracking system software may utilize the digital camera 18 to identify and track tissue as it is sectioned from the original sample and placed into tissue containers by recognizing the unique tissue characteristics, such as but not limited to cell morphology, shape, etc., thus, the system may verify and confirm the tissue sample is properly matched with the correct sample container throughout the dissection process. The system 34 then provides continuous monitoring of the chain of custody of the tissue samples from the moment of dissection and makes sure that the tissue remains in its original container 46 or associated dissection labeled container 44. This process eliminates reliance on humans and the possibility of human error and provides a high level of security in sample tracking during the dissection procedures. The system 34 may also employ machine learning or AI to help identify known cell morphologies and recommend dissection grid patterns for the original sampled tissue 40, thus ensuring optimal tissue sectioning for the histology performed thereon. This gross dissection sample tracking system 34 operates from the initial dissection step and provides continuous confirmation and monitoring of the tissues location, such that it is matched with its containers throughout the entire process and keeps the overall integrity of the dissection process reliable.

The present invention has been described in an illustrative manner. It is to be understood that the terminology, which has been used is intended to be in the nature of words of description rather than that of limitation.

Many modifications and variations of the present invention are possible in light of the above teachings. Therefore, within the scope of the appended claims, the present invention may be practiced otherwise than as specifically described.

What is claimed is:

1. A gross dissection sample tracking system for use on a grossing station having a gross dissection process performed thereon, said system comprising:
a software application that identifies a specific tissue section and associates it with an identified and properly labeled sample container; and
said association ensures that said specific tissue section remains in said labeled sample container throughout the gross dissection process.

2. The gross dissection sample tracking system of claim 1 further comprising a dissection table.

3. The gross dissection sample tracking system of claim 1 further comprising a digital camera arranged on a surface of said grossing station.

4. The gross dissection sample tracking system of claim 3, wherein said digital camera captures images or continuous video of the gross dissection process.

5. The gross dissection sample tracking system of claim 1, wherein the software application tracks unique characteristics or cell morphology of said specific tissue section cut from an original biopsy tissue sample.

6. The gross dissection sample tracking system of claim 5, wherein said labeled sample container having a unique barcode, 2D data matrix, QR code or other identification label arranged on a surface thereof.

7. The gross dissection sample tracking system of claim 6, wherein said specific tissue section is always confirmed to be correctly mated to said specific sample container and remains therein to provide chain of custody from the moment it is dissected from said original biopsy tissue sample.

8. The gross dissection sample tracking system of claim 1, wherein said specific tissue section is identified by a tissue section shape and by tissue morphology characteristics, a plurality of another specific tissue sections is cut from an original biopsy tissue sample and placed in and associated with one of a plurality of another labeled sample containers.

9. The gross section sample tracking system of claim 8, wherein said tissue section shape and said tissue morphology characteristics are mapped and associated with said labeled sample container.

10. The gross dissection sample tracking system of claim 1, wherein said software application recommends an appropriate dissection pattern for an original biopsied tissue sample based on its morphology.

11. The gross dissection sample tracking system of claim 10, wherein the software application continuously monitors and confirms that said specific tissue section matches a recommended pattern and said labeled sample container identification label.

12. The gross dissection sample tracking system of claim 1, wherein said software application utilizes machine learning or artificial intelligence to identify known cell morphologies and optimize a dissection pattern for an original biopsied tissue sample in coordination with industry or scientific guidelines to ensure said original biopsied tissue sample is sectioned in the most appropriate manner.

13. A method of tracking original biopsied tissue samples during a gross dissection process, said method comprising the steps of:
capturing a continuous digital camera feed of the dissection area;
analyzing tissue characteristics;
associating said original biopsy tissue sample with a labeled container; and
maintaining a real time chain of custody from an initial point of dissection to a final processing stage.

14. The method of claim 13, wherein said step of capturing having a gross dissection table and a digital camera arranged on a surface of said gross dissection table.

15. The method of claim 14, wherein said step of capturing takes images or video via said digital camera.

16. The method of claim 13, wherein the step of analyzing having a software application that identifies and tracks a plurality of tissue dissection samples unique characteristics or cell morphology.

17. The method of claim 16, wherein the step of associating identifies an original sample container via a barcode, 2D data matrix, QR code or other identification label.

18. The method of claim 17, wherein the step of associating mates forever said one of said plurality of said tissue dissection samples with said original biopsy tissue sample or associated with one of a plurality of labeled sample containers to provide proper chain of custody from the moment of dissection.

19. The method of claim 16, wherein said software application utilizes machine learning or artificial intelligence to identify known cell morphologies and recommend a dissection sample grid pattern.

20. A gross dissection sample tracking system, comprising:
a digital camera for capturing images or video of the dissection process;
a software application configured to detect and analyze tissue characteristics;
said software application matches said tissue to a labeled sample container;
said software application continuously monitors said tissue location and association with said labeled sample container;
said software application continuously tracks and monitors said tissue location and association with said labeled container; and
the system provides a chain of custody tracking without human intervention, which reduces or eliminates gross dissection workflow errors.
